(19) United States
(12) Patent Application Publication
(10) Pub. No.: US 2025/0302869 A1
(43) Pub. Date: Oct. 2, 2025

(54) USE OF INHALED NITRIC OXIDE (INO) FOR TREATING PATIENTS WITH PULMONARY HYPERTENSION ASSOCIATED WITH SARCOIDOSIS (PH-SARC)

(71) Applicant: Mallinckrodt Pharmaceuticals Ireland Limited, Dublin (IR)

(72) Inventors: Parag SHAH, MORRISTOWN, NJ (US); Peter Paul FERNANDES, RANDOLPH, NJ (US); Bobae KIM, MORRISTOWN, NJ (US); Martin DEKKER, CALIFON, NJ (US)

(21) Appl. No.: 18/726,314

(22) PCT Filed: Jan. 4, 2023
(86) PCT No.: PCT/US2023/060080

§ 371 Date: Jul. 2, 2024

Related U.S. Application Data
(60) Provisional application No. 63/296,359, filed on Jan. 4, 2022.
(60) Provisional application No. 63/341,986, filed on May 13, 2022.
(60) Provisional application No. 63/342,535, filed on May 16, 2022.

(51) Int. Cl. A61K 33/00; A61K 9/00; A61P 9/12

(52) U.S. Cl. CPC A61K 33/00; A61K 9/0073; A61P 9/12

(57) ABSTRACT

Described are methods for treating patients with pulmonary hypertension associated with sarcoidosis (PH-SARC) using inhaled nitric oxide (iNO).

### CROSS REFERENCE TO RELATED APPLICATIONS

This application claims the benefit of U.S. Provisional Patent Application No. 63/296,359, filed Jan. 4, 2022, entitled “Use of Inhaled Nitric Oxide for Decreasing Pulmonary Arterial Pressure and Pulmonary Vascular Resistance,” U.S. Provisional Application No. 63/341,986, filed May 13, 2022, entitled “Use of Inhaled Nitric Oxide for Decreasing Pulmonary Arterial Pressure and Pulmonary Vascular Resistance,” U.S. Provisional Application No. 63/342,535, filed May 16, 2022, entitled “Use of Inhaled Nitric Oxide for Decreasing Pulmonary Arterial Pressure and Pulmonary Vascular Resistance,” all of which are incorporated by reference herein in their entirety.

### FIELD

The present application relates generally to apparatus and methods for administration of nitric oxide, in some embodiments, pulsatile delivery of nitric oxide to patients having pulmonary hypertension associated with sarcoidosis (PH-SARC), and also relates generally to methods for administration of nitric oxide, in some embodiments, pulsatile delivery of nitric oxide to same patients to decrease pulmonary arterial pressure (PAP) and pulmonary vascular resistance (PVR).

### BACKGROUND

Nitric oxide (NO) is a gas that, when inhaled, acts to dilate blood vessels in the lungs, improving oxygenation of the blood and reducing pulmonary hypertension. Because of this, nitric oxide is provided as a therapeutic gas in the inspiratory breathing phase for patients having shortness of breath (dyspnea), fatigue, reduced exercise capacity, oxygen desaturation, as well as potentially other indications due to a disease state, for example, pulmonary arterial hypertension (PAH), chronic obstructive pulmonary disease (COPD), combined pulmonary fibrosis and emphysema (CPFE), cystic fibrosis (CF), idiopathic pulmonary fibrosis (IPF), emphysema, interstitial lung disease (ILD), chronic thromboembolic pulmonary hypertension (CTEPH), chronic high altitude sickness, or other lung disease.

While NO may be therapeutically effective when administered under the appropriate conditions, it can also become toxic if not administered correctly. NO reacts with oxygen to form nitrogen dioxide (NO₂), and NO₂ can be formed when oxygen or air is present in the NO delivery conduit. NO₂ is a toxic gas which may cause numerous side effects, and the Occupational Safety & Health Administration (OSHA) provides that the permissible exposure limit for general industry is only 5 ppm. Thus, it is desirable to limit exposure to NO₂ during NO therapy.

### BRIEF DESCRIPTION OF THE DRAWINGS

The following detailed description of embodiments of the use of iNO for treating patients with pulmonary hypertension associated PH-SARC, will be better understood when read in conjunction with the appended drawings of an exemplary embodiments. It should be understood, however, that the disclosure is not limited to the precise arrangements and instrumentalities shown.

In the drawings:

FIG. 1 is a schematic demonstrating an exemplary acute iNO dose escalation study design, including examination of changes in mPAP, PCWP, CO and PVR across various doses inhaled nitric oxide (iNO).

FIGS. 2A-2H are graphs of experimental data demonstrating the absolute change from baseline hemodynamics as related to changes in PVR, mPAP, CO, and PCWP.

### DETAILED DESCRIPTION

Unless defined otherwise, all technical and scientific terms used herein have the same meaning as is commonly understood by one of skill in the art to which this disclosure belongs.

Before describing several exemplary embodiments of the disclosure, it is to be understood that the disclosure is not limited to the details of construction or process steps set forth in the following description. The disclosure is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

### Definitions

The term “effective amount” or “therapeutically effective amount” refers to that amount of a compound or combination of compounds as described herein that is sufficient to affect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof

When ranges are used herein to describe an aspect of the present disclosure, for example, dosing ranges, amounts of a component of a formulation, etc., all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to about 25%, 0% to about 20%, 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of or “consist essentially of the described features.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus, such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

As used herein, the terms “pulmonary hypertension associated with sarcoidosis (PH-SARC)” and “sarcoidosis associated pulmonary hypertension (SAPH)” are used interchangeably.

Sarcoidosis is characterized by the growth of inflammatory cells (granulomas) most commonly in the lungs or lymphatic tissues. The cause of sarcoidosis is not known but is believed to be an immune reaction to an unknown trigger such as infection or chemical in those that are genetically predisposed. Symptoms include fatigue, weight loss, joint aches and pains, dry eyes, swelling of the knees, blurry vision, shortness of breath, a dry, hacking cough, or skin lesions.

With respect to the present disclosure, in certain embodiments, a dose of a gas (e.g., NO) is administered to a patient during an inspiration by the patient. In embodiments, the dose of a gas (e.g., NO) is administered in a pulse to a patient during an inspiration by the patient. It has been surprisingly discovered that nitric oxide delivery can be precisely and accurately delivered over a total breath inspiration time or a portion thereof, for example over the first two-thirds of total breath inspiration time, and the patient obtains benefits from such delivery. Such delivery minimizes loss of drug product and risk of detrimental side effects increases the efficacy of a pulse dose which in turn results in a lower overall amount of NO that needs to be administered to the patient in order to be effective. Such delivery is useful for the treatment of various diseases, such as but not limited to pulmonary hypertension associated with sarcoidosis (PH-SARC), including World Health Organization (WHO) Group I-V pulmonary hypertension.

Effective dosing of NO is based on a number of different variables, including quantity of drug and the timing of delivery. Several patents have been granted relating to NO delivery, including U.S. Pat. Nos. 7,523,752; 8,757,148; 8,770,199; and 8,803,717, and a Design Patent D701,963 for a design of an NO delivery device, all of which are herein incorporated by reference. Additionally, there are pending applications relating to delivery of NO, including US2013/0239963 and US2016/0106949, both of which are herein incorporated by reference. Even in view of these patents and pending publications, there is still a need for methods and apparatuses that deliver NO in a precise, controlled manner, so as to maximize the benefit of a therapeutic dose and minimize the potentially harmful side effects for the treatment of pulmonary hypertension associated with sarcoidosis (PH-SARC).

Such precision has further advantages in that only portions of the poorly ventilated lung area is exposed to NO. Hypoxia and issues with hemoglobin may also be reduced with such pulsed delivery, while NO₂ exposure is also more limited.

### A Device of the Present Disclosure

In certain embodiments, the present disclosure includes a device, e.g., a programmable device for delivering a dose of a gas (e.g., nitric oxide) to a patient in need. The device can include a delivery portion, a drug cartridge including a compressed gas for delivery to a patient, a breath sensitivity portion to detect a breath pattern in patient comprising a breath sensitivity setting, at least one breath detection algorithm for determining when to administer the compressed gas to the patient and a portion for administering the dose of nitric oxide to the patient through a series of one or more pulses.

In certain embodiments, the drug cartridge is replaceable.

In certain embodiments, the delivery portion includes one or more of a nasal cannula, a face mask, an atomizer, and a nasal inhaler. In certain embodiments, the delivery portion can further include a second delivery portion to permit the simultaneous administration of one or more other gases (e.g., oxygen) to a patient.

In certain embodiments, and as detailed elsewhere herein, the device includes an algorithm wherein the algorithm uses one or both of a threshold sensitivity and a slope algorithm, wherein the slope algorithm detects a breath when the rate of pressure drop reaches a predetermined threshold.

In an embodiment of the disclosure, mechanically, a pulse dose of a gas can reduce, if not eliminate, venturi effects which would normally create problems for other gas sensors. For example, in the absence of the pulse doses of the present disclosure, O₂ back pressure sensors may override delivery of O₂ when O₂ is administered simultaneously with another gas such as NO.

### Breath Patterns, Detection and Triggers

Breath patterns vary based on the individual, time of day, level of activity, and other variables; thus, it is difficult to predetermine a breath pattern of an individual. A delivery system that delivers therapeutics to a patient based on breath pattern, then, should be able to handle a range of potential breath patterns in order to be effective.

In certain embodiments, the patient or individual can be any age, however, in more certain embodiments the patient is sixteen years of age or older or eighteen years of age or older.

In an embodiment of the disclosure, the breath pattern includes a measurement of total inspiratory time, which as used herein is determined for a single breath. However, depending on context “total inspiratory time” can also refer to a summation of all inspiratory times for all detected breaths during a therapy. Total inspiratory time may be observed or calculated. In another embodiment, total inspiratory time is a validated time based on simulated breath patterns.

In an embodiment of the disclosure, breath detection includes at least one, and in some embodiments, at least two separate triggers functioning together, namely a breath level trigger and/or a breath slope trigger.

In an embodiment of the disclosure, a breath level trigger algorithm is used for breath detection. The breath level trigger detects a breath when a threshold level of pressure (e.g., a threshold negative pressure) is reached upon inspiration.

In an embodiment of the disclosure, a breath slope trigger detects breath when the slope of a pressure waveform indicates inspiration. The breath slope trigger is, in certain instances, more accurate than a threshold trigger, particularly when used for detecting short, shallow breaths.

In an embodiment of the disclosure, a combination of these two triggers provides overall a more accurate breath detection system, particularly when multiple therapeutic gases are being administered to a patient simultaneously.

In an embodiment of the disclosure, the breath sensitivity control for detection of either breath level and/or breath slope is fixed. In an embodiment of the disclosure, the breath sensitivity control for detection of either breath level or breath slope is adjustable or programmable. In an embodiment of the disclosure, the breath sensitivity control for either breath level and/or breath slope is adjustable from a range of least sensitive to most sensitive, whereby the most sensitive setting is more sensitive at detecting breaths than the least sensitive setting.

In certain embodiments where at least two triggers are used, the sensitivity of each trigger is set at different relative levels. In one embodiment where at least two triggers are used, one trigger is set a maximum sensitivity and another trigger is set at less than maximum sensitivity. In one embodiment where at least two triggers are used and where one trigger is a breath level trigger, the breath level trigger is set at maximum sensitivity.

Oftentimes, not every inhalation/inspiration of a patient is detected to then be classified as an inhalation/inspiration event for the administration of a pulse of gas (e.g., NO). Errors in detection can occur, particularly when multiple gases are being administered to a patient simultaneously, e.g., NO and oxygen combination therapies.

Embodiments of the present disclosure, and in particular an embodiment which incorporates a breath slope trigger alone or in combination with another trigger, can maximize the correct detection of inspiration events to thereby maximize the effectiveness and efficiency of a therapy while also minimizing waste due to misidentification or errors in timing.

In certain embodiments, greater than 50% of the total number of inspirations of a patient over a timeframe for gas delivery to the patient are detected. In certain embodiments, greater than 75% of the total number of inspirations of a patient are detected. In certain embodiments, greater than 90% of the total number of inspirations of a patient are detected. In certain embodiments, greater than 95% of the total number of inspirations of a patient are detected. In certain embodiments, greater than 98% of the total number of inspirations of a patient are detected. In certain embodiments, greater than 99% of the total number of inspirations of a patient are detected. In certain embodiments, 75% to 100% of the total number of inspirations of a patient are detected.

### Dosages and Dosing Regimens

In an embodiment of the disclosure, nitric oxide delivered to a patient is formulated at concentrations of about 3 to about 18 mg NO per liter, about 6 to about 10 mg per liter, about 3 mg NO per liter, about 6 mg NO per liter, about 15 mg NO per liter, or about 18 mg NO per liter. The NO may be administered alone or in combination with an alternative gas therapy. In certain embodiments, oxygen (e.g., concentrated oxygen) can be administered to a patient in combination with NO. In embodiments, the NO is inhaled nitric oxide (iNO).

In an embodiment of the present disclosure, a volume of nitric oxide is administered (e.g., in a single pulse) in an amount of from about 0.350 mL to about 7.5 mL per breath. In some embodiments, the volume of nitric oxide in each pulse dose may be identical during the course of a single session. In some embodiments, the volume of nitric oxide in some pulse doses may be different during a single timeframe for gas delivery to a patient. In some embodiments, the volume of nitric oxide in each pulse dose may be adjusted during the course of a single timeframe for gas delivery to a patient as breath patterns are monitored. In an embodiment of the disclosure, the quantity of nitric oxide (in ng) delivered to a patient for purposes of treating or alleviating symptoms of a pulmonary disease on a per pulse basis (the “pulse dose”) is calculated as follows and rounded to the nearest nanogram value:

$$Dose mcg / kg - IBW / hr \times Ideal body weight in kg ( kg - IBW ) \times ( ( 1 hr / 60 min ) \times ( 1 min / respiratory rate ( bpm ) ) \times ( 1 , 000 ng / ug ) .$$

As an example, Patient A at a dose of 100 mcg/kg IBW/hr has an ideal body weight of 75 kg, has a respiratory rate of 20 breaths per minute (or 1200 breaths per hour):

$$100 mcg / kg - IBW / hr \times 75 kg \times ( 1 hr / 1200 breaths ) \times ( 1 , 000 ng / ug ) = 6250 ng per pulse$$

In certain embodiments, the 60/respiratory rate (ms) variable may also be referred to as the Dose Event Time. In another embodiment of the disclosure, a Dose Event Time is 1 second, 2 seconds, 3 seconds, 4 seconds, 5 seconds, 6 seconds, 7 seconds, 8 seconds, 9 seconds, or 10 seconds.

In an embodiment of the disclosure, a single pulse dose provides a therapeutic effect (e.g., a therapeutically effective amount of NO) to the patient. In another embodiment of the disclosure, an aggregate of two or more pulse doses provides a therapeutic effect (e.g., a therapeutically effective amount of NO) to the patient.

In an embodiment of the disclosure, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 675, about 700, about 750, about 800, about 850, about 900, about 950, or about 1000 pulses of nitric oxide is administered to a patient every hour.

In an embodiment of the disclosure, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the disclosure, a nitric oxide treatment is administered for a timeframe of a minimum course of treatment. In an embodiment of the disclosure, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the disclosure, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the disclosure, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the disclosure, a nitric oxide treatment session is administered one or more times per day. In an embodiment of the disclosure, nitric oxide treatment session may be once, twice, three times, four times, five times, six times, or more than six times per day. In an embodiment of the disclosure, the treatment session may be administered once a month, once every two weeks, once a week, once every other day, daily, or multiple times in one day.

### Timing of a Pulse of NO

In an embodiment of the disclosure, the breath pattern is correlated with an algorithm to calculate the timing of administration of a dose of nitric oxide.

The precision of detection of an inhalation/inspiration event also permits the timing of a pulse of gas (e.g., NO) to maximize its efficacy by administering gas at a specified time frame of the total inspiration time of a single detected breath.

In an embodiment of the disclosure, at least fifty percent (50%) of the pulse dose of a gas is delivered over the first third of the total inspiratory time of each breath. In an embodiment of the disclosure, at least sixty percent (60%) of the pulse dose of a gas is delivered over the first third of the total inspiratory time. In an embodiment of the disclosure, at least seventy-five percent (75%) of the pulse dose of a gas is delivered over the first third of the total inspiratory time for each breath. In an embodiment of the disclosure, at least eighty-five (85%) percent of the pulse dose of a gas is delivered over the first third of the total inspiratory time for each breath. In an embodiment of the disclosure, at least ninety percent (90%) of the pulse dose of a gas is delivered over the first third of the total inspiratory time. In an embodiment of the disclosure, at least ninety-two percent (92%) of the pulse dose of a gas is delivered over the first third of the total inspiratory time. In an embodiment of the disclosure, at least ninety-five percent (95%) of the pulse dose of a gas is delivered over the first third of the total inspiratory time. In an embodiment of the disclosure, at least ninety-nine (99%) of the pulse dose of a gas is delivered over the first third of the total inspiratory time. In an embodiment of the disclosure, 90% to 100% of the pulse dose of a gas is delivered over the first third of the total inspiratory time.

In an embodiment of the disclosure, at least seventy percent (70%) of the pulse dose is delivered to the patient over the first half of the total inspiratory time. In yet another embodiment, at least seventy-five percent (75%) of the pulse dose is delivered to the patient over the first half of the total inspiratory time. In an embodiment of the disclosure, at least eighty percent (80%) of the pulse dose is delivered to the patient over the first half of the total inspiratory time. In an embodiment of the disclosure, at least 90 percent (90%) of the pulse dose is delivered to the patient over the first half of the total inspiratory time. In an embodiment of the disclosure, at least ninety-five percent (95%) of the pulse dose is delivered to the patient over the first half of the total inspiratory time. In an embodiment of the disclosure, 95% to 100% of the pulse dose of a gas is delivered over the first half of the total inspiratory time

In an embodiment of the disclosure, at least ninety percent (90%) of the pulse dose is delivered over the first two-thirds of the total inspiratory time. In an embodiment of the disclosure, at least ninety-five percent (95%) of the pulse dose is delivered over the first two-thirds of the total inspiratory time. In an embodiment of the disclosure, 95% to 100% of the pulse dose is delivered over the first two-thirds of the total inspiratory time.

When aggregated, administration of a number of pulse doses over a therapy session/timeframe can also meet the above ranges. For example, when aggregated greater than 95% of all the pulse doses administered during a therapy session were administered over the first two thirds of all of the inspiratory times of all of the detected breaths. In higher precision embodiments, when aggregated greater than 95% of all the pulse doses administered during a therapy session were administered over the first third of all of the inspiratory times of all of the detected breaths.

Given the high degree of precision of the detection methodologies of the present disclosure, a pulse dose can be administered during any specified time window of an inspiration. For example, a pulse dose can be administered targeting the first third, middle third or last third of a patient's inspiration. Alternatively, the first half or second half of an inspiration can be targeted for pulse dose administration. Further, the targets for administration may vary. In one embodiment, the first third of an inspiration time can be targeted for one or a series of inspirations, where the second third or second half may be targeted for one or a series of subsequent inspirations during the same or different therapy session. Alternatively, after the first quarter of an inspiration time has elapsed the pulse dose begins and continues for the middle half (next two quarters) and can be targeted such that the pulse dose ends at the beginning of the last quarter of inspiration time. In some embodiments, the pulse may be delayed by 50, 100, or 200 milliseconds (ms) or a range from about 50 to about 200 milliseconds.

The utilization of a pulsed dose during inhalation reduces the exposure of poorly ventilated areas of the lung and alveoli from exposure to a pulsed dose gas, e.g., NO. In one embodiment, less than 5% of poorly ventilated (a) areas of the lung or (b) alveoli are exposed to NO. In one embodiment, less than 10% of poorly ventilated (a) areas of the lung or (b) alveoli are exposed to NO. In one embodiment, less than 15% of poorly ventilated (a) areas of the lung or (b) alveoli are exposed to NO. In one embodiment, less than 20% of poorly ventilated (a) areas of the lung or (b) alveoli are exposed to NO. In one embodiment, less than 25% of poorly ventilated (a) areas of the lung or (b) alveoli are exposed to NO. In one embodiment, less than 30% of poorly ventilated (a) areas of the lung or (b) alveoli are exposed to NO. In one embodiment, less than 50% of poorly ventilated (a) areas of the lung or (b) alveoli are exposed to NO. In one embodiment, less than 60% of poorly ventilated (a) areas of the lung or (b) alveoli are exposed to NO. In one embodiment, less than 70% of poorly ventilated (a) areas of the lung or (b) alveoli are exposed to NO. In one embodiment, less than 80% of poorly ventilated (a) areas of the lung or (b) alveoli are exposed to NO. In one embodiment, less than 90% of poorly ventilated (a) areas of the lung or (b) alveoli are exposed to NO.

### Methods of Treatment

In one aspect of the disclosure, methods for treating pulmonary hypertension associated with sarcoidosis (PH-SARC) in a patient in need thereof are described. In another embodiment, methods for reducing pulmonary vascular resistance (PVR) in a patient having or at risk of having and/or developing pulmonary hypertension associated with sarcoidosis (PH-SARC), for example compared to baseline levels, are provided. In another embodiment, reducing mean pulmonary artery pressure (mPAP) in a patient having or at risk of having and/or developing pulmonary hypertension associated with sarcoidosis (PH-SARC), for example compared to baseline levels, are provided. In some embodiments, the method includes comparing the PVR and/or mPAP to baseline levels. In some embodiments, the methods include administration of iNO, optionally supplementing iNO administration with oxygen. In an embodiment of the disclosure, iNO is administered according to the pulsed manner discussed herein. In an embodiment of the disclosure, the iNO is delivered to a patient using the INOpulse® device (Bellerophon Therapeutics). In an embodiment, the patient has or is at risk of having and/or developing pulmonary hypertension associated with sarcoidosis (PH-SARC). In an embodiment, the patient is at low risk of having and/or developing pulmonary hypertension associated with sarcoidosis (PH-SARC). In an embodiment, the patient is at intermediate risk of having and/or developing pulmonary hypertension associated with sarcoidosis (PH-SARC). In an embodiment, the patient is at high risk of having and/or developing pulmonary hypertension associated with sarcoidosis (PH-SARC). In an embodiment, the patient has or is at risk of having and/or developing pulmonary hypertension (PH). In an embodiment, the patient is at low risk of having and/or developing pulmonary hypertension (PH). In an embodiment, the patient is at intermediate risk of having and/or developing pulmonary hypertension (PH). In an embodiment, the patient is at high risk of having and/or developing pulmonary hypertension (PH). In some embodiments, the pulmonary hypertension is selected from WHO Group I, WHO Group II, WHO Group III, WHO Group IV, and WHO Group V pulmonary hypertension. In some embodiments, the pulmonary hypertension is WHO Group V pulmonary hypertension.

In one embodiment, the patient is administered iNO for a period of at least about 12 hours, 13 hours, 14, hours, 15 hours, 16 hours, 17 hours, 18 hours, 19 hours, 20 hours, 21 hours, 22 hours, 23 hours, or 24 hours per day for a period of at least about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks or 20 weeks. In one embodiment, the patient is administered iNO for 8 weeks. In another embodiment, the patient is administered iNO for 16 weeks. In an embodiment of the disclosure, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the disclosure, a nitric oxide treatment is administered for a timeframe of a minimum course of treatment. In an embodiment of the disclosure, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the disclosure, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the disclosure, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In one embodiment of the disclosure, the iNO is administered at a dose ranging from about 10 mcg/kg ideal body weight (MW)/hr to about 200 mcg/kg IBW/hr or more. In one embodiment, the iNO is administered a dose ranging from about 20 mcg/kg IBW/hr to about 150 mcg/kg MW/hr. In one embodiment, the iNO is administered a dose ranging from about 25 mcg/kg IBW/hr to about 100 mcg/kg IBW/hr. In one embodiment, the iNO is administered a dose ranging from about 30 mcg/kg IBW/hr to about 75 mcg/kg IBW/hr. In one embodiment, the iNO is administered a dose ranging a dose ranging from about 25 mcg/kg IBW/hr to about 50 mcg/kg IBW/hr. In one embodiment, the iNO is administered a dose ranging from about 30 mcg/kg IBW/hr to about 45 mcg/kg IBW/hr. In one embodiment, the iNO is administered at a dose of about 25 mcg/kg MW/hr. In one embodiment, the iNO is administered at a dose of about 30 mcg/kg IBW/hr. In one embodiment, the iNO is administered at a dose of about 35 mcg/kg IBW/hr. In one embodiment, the iNO is administered at a dose of about 40 mcg/kg IBW/hr. In one embodiment, the iNO is administered at a dose of about 45 mcg/kg IBW/hr. In one embodiment, the iNO is administered at a dose of about 50 mcg/kg IBW/hr. In one embodiment, the iNO is administered at a dose of about 55 mcg/kg IBW/hr. In one embodiment, the iNO is administered at a dose of about 60 mcg/kg IBW/hr. In one embodiment, the iNO is administered at a dose of about 65 mcg/kg IBW/hr. In one embodiment, the iNO is administered at a dose of about 70 mcg/kg IBW/hr. In one embodiment, the iNO is administered at a dose of about 75 mcg/kg IBW/hr. In one embodiment, the iNO is administered at a dose of about 80 mcg/kg IBW/hr. In one embodiment, the iNO is administered at a dose of about 85 mcg/kg IBW/hr. In one embodiment, the iNO is administered at a dose of about 90 mcg/kg IBW/hr. In one embodiment, the iNO is administered at a dose of about 95 mcg/kg IBW/hr. In one embodiment, the iNO is administered at a dose of about 100 mcg/kg IBW/hr. In one embodiment, the iNO is administered at a dose of about 105 mcg/kg IBW/kg. In one embodiment, the iNO is administered at a dose of about 110 mcg/kg IBW/hr. In one embodiment, the iNO is administered at a dose of about 115 mcg/kg IBW/hr. In one embodiment, the iNO is administered at a dose of about 120 mcg/kg IBW/hr. In one embodiment, the iNO is administered at a dose of about 125 mcg/kg IBW/hr. In one embodiment, the iNO is administered at a dose of about 130 mcg/kg IBW/hr. In one embodiment, the iNO is administered at a dose of about 135 mcg/kg IBW/hr. In one embodiment, the iNO is administered at a dose of about 140 mcg/kg IBW/hr. In one embodiment, the iNO is administered at a dose of about 145 mcg/kg IBW/hr. In one embodiment, the iNO is administered at a dose of about 150 mcg/kg IBW/hr. In one embodiment, the iNO is administered at a dose of about 155 mcg/kg IBW/hr. In one embodiment, the iNO is administered at a dose of about 160 mcg/kg IBW/hr. In one embodiment, the iNO is administered at a dose of about 165 mcg/kg IBW/hr. In one embodiment, the iNO is administered at a dose of about 170 mcg/kg IBW/hr. In one embodiment, the iNO is administered at a dose of about 175 mcg/kg IBW/hr. In one embodiment, the iNO is administered at a dose of about 180 mcg/kg IBW/hr. In one embodiment, the iNO is administered at a dose of about 185 mcg/kg IBW/hr. In one embodiment, the iNO is administered at a dose of about 190 mcg/kg IBW/hr. In one embodiment, the iNO is administered at a dose of about 195 mcg/kg IBW/hr. In one embodiment, the iNO is administered at a dose of about 200 mcg/kg IBW/hr.

In an embodiment of the disclosure, the patient is also administered oxygen with the iNO. In an embodiment of the disclosure, the oxygen is administered at up to 20 L/minute. In an embodiment of the disclosure, the oxygen is administered at up to 1 L/minute, 2 L/minute, 3 L/minute, 4 L/minute, 5 L/minute, 6 L/minute, 7 L minute, 8 L/minute, 9 L/minute, 10 L/minute, 11 L/minute, 12 L/minute, 13 L/minute, 14 L/minute, 15 L/minute, 16 L/minute, 17 L/minute, 18 L/minute, 19 L/minute, or 20 L/minute. In an embodiment of the disclosure, oxygen is administered as prescribed by a physician. In some embodiments, the patient is undergoing long term oxygen therapy (LTOT).

In some embodiments, the method for treating pulmonary hypertension associated with sarcoidosis (PH-SARC) in a patient in need thereof further comprises treating and/or reducing the severity of one or more symptoms associated with PH-SARC. Non-limiting examples of symptoms include reducing pulmonary vascular resistance (PVR) and reducing mean pulmonary artery pressure (mPAP), wherein the symptoms are reduced compared to baseline levels. In some embodiments, the method for treating pulmonary hypertension associated with sarcoidosis (PH-SARC) in a patient in need thereof further comprises maintaining the severity of one or more symptoms associated with PH-SARC. Non-limiting examples of symptoms include reducing pulmonary vascular resistance (PVR) and reducing mean pulmonary artery pressure (mPAP)

In a non-limiting embodiment, baseline levels can be determined by measuring and/or calculating the level (e.g. PVR, mPAP) in a patient or a cohort of patients (e.g. one or more patients having PH-SARC) prior to the administration of iNO.

In some embodiments, pulmonary vascular resistance (PVR) is reduced by at least about 5%, about 10%, about 15%, about 20%, about 25%, about 30%, about 35%, about 40%, about 45%, or about 50% or more compared to baseline levels. In some embodiments, pulmonary vascular resistance (PVR) is reduced by at least about 20% compared to baseline levels.

In some embodiments, mean pulmonary artery pressure (mPAP) is reduced by at least about 5%, about 10%, about 15%, about 20%, about 25%, about 30%, about 35%, about 40%, about 45%, or about 50% or more compared to baseline levels. In some embodiments, mean pulmonary artery pressure (mPAP) is reduced by at least about 10% compared to baseline levels.

In one aspect, the methods for treating pulmonary hypertension associated with sarcoidosis (PH-SARC) in a patient in need thereof disclosed herein do not cause or result in the incidence of treatment emergent adverse events (AEs) and/or provide a reduction in severity of treatment emergent adverse events (AEs) compared to other forms of treatment. In some embodiments, the treatment emergent adverse events (AEs), including those related to device deficiency.

In embodiments, the methods for treating pulmonary hypertension associated with sarcoidosis (PH-SARC) in a patient in need thereof disclosed herein do not cause or result in symptoms that may be due to rebound associated with acute withdrawal of iNO and/or provide a reduction in severity of symptoms that may be due to rebound associated with acute withdrawal of iNO compared to other nitric oxide treatments. Non-limiting examples of symptoms that may be due to rebound associated with acute withdrawal of iNO: systemic arterial oxygen desaturation, hypoxemia, bradycardia, tachycardia, systemic hypotension, shortness of breath, near-syncope, and syncope.

In embodiments, the methods for treating pulmonary hypertension associated with sarcoidosis (PH-SARC) in a patient in need thereof disclosed herein result in the maintenance of vital signs and/or other parameters, for example compared to baseline levels. In embodiments, the vital signs and/or other parameters are adversely affected by the administration of other types of treatment compared to baseline levels. Non-limiting examples of other parameters include a change in oxygen saturation, cardiac output (CO), and pulmonary capillary wedge pressure (PCWP). In embodiments, the method further comprises maintaining a resting cardiac output (CO) compared to baseline levels. In embodiments, the method further comprises maintaining pulmonary capillary wedge pressure (PCWP) compared to baseline levels.

In an embodiment of the disclosure, other parameters useful in assessing the effects of iNO include time to clinical improvement and time to clinical worsening. A shortening of the time it takes to see clinical improvement and a lengthening of the time it takes to see clinical worsening is expected in patients treated with iNO. Patient related outcome measurements (PROs) are also useful in assessing the effects of iNO. PROs are measured in the form of questionnaires, which provide a subject's perspective on overall quality of life. Non-limiting examples of PROs include the St. George's Respiratory Questionnaire (SGRQ) and the University of California, San Diego Shortness of Breath Questionnaire (UC SD SOBQ), Kings Sarcoidosis Questionnaire (KSQ), Fatigue Assessment Scale (FAS), and emPHasis 10. These Questionnaires are standard questionnaires used in the art and are well-known and clinically accepted. An improvement in scores for both of these PROs is expected for patients on iNO therapy.

### Actigraphy

The present disclosure also relates to methods of improving or maintaining activity levels, or preventing a decline in activity levels, in patients having or at risk of having and/or developing pulmonary hypertension associated with sarcoidosis (PH-SARC). The methods include using actigraphy to monitor and measure changes in activity level.

In embodiments, actigraphy involves use of a wearable activity monitor, similar to a pedometer or a accelerometer, or a triaxial accelerometer, an Actigraph GT9X, or a FITBIT® that measures activity parameters. Such activity monitor assesses activity and measures activity parameters of the user. Activity parameters measured include overall activity, non-sedentary activity, moderate activity, moderate to vigorous physical activity (MVPA), steps, calories, metabolic equivalent units (MET), sleep, heart rate, oxygen saturation, calories burned, six-minute walk distance (6MWD) test, and other types of activity and/or daily activity parameters.

In an embodiment of the disclosure, activity levels are monitored and measured constantly over a period of time. In an embodiment of the disclosure, activity levels are monitored and measured intermittently over a period of time. In one embodiment, activity levels are monitored and measured for a period of at least about 8 hours, 9 hours, 10 hours, 11 hours, 12 hours, 13 hours, 14, hours, 15 hours, 16 hours, 17 hours, 18 hours, 19 hours, 20 hours, 21 hours, 22 hours, 23 hours, or 24 hours per day for a period of at least about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, or 26 weeks. In another embodiment, activity levels are monitored and measured for a period of at least about 8 hours, 9 hours, 10 hours, 11 hours, 12 hours, 13 hours, 14, hours, 15 hours, 16 hours, 17 hours, 18 hours, 19 hours, 20 hours, 21 hours, 22 hours, 23 hours, or 24 hours per day for a period of at least about 1 month, 2 months, 3 months, 4 months, 5 months, or 6 months. In another embodiment, activity levels are monitored only during times when the patient is awake. In one embodiment, activity levels are measured in a continuous manner over the entire awake time period. In another embodiment, patients may remove the device for certain activities, thus activity levels are measured in a non-continuous manner over the awake time period. In another embodiment, the awake time period is at least 10 hours. In another embodiment, the awake time period is at least 8 hours. In another embodiment, the awake time period is at least 12 hours. In another embodiment, the awake time period is at least 14 hours.

In an embodiment of the disclosure, activity levels are improved as compared with a baseline activity level. In an embodiment, the baseline activity level is monitored and measured for at least one week prior to administration of vasodilators. In another embodiment, baseline activity level is monitored or measured for about 1 day to about 14 days, for about 1 day to about 10 days, for about 1 day to about 7 days, or for about 1 day to about 5 days. In another embodiment, baseline activity level is monitored or measured for about 1 day, 2 days, 3 days, 4 days, 5 days, 6 days, 7 days, 8 days, 9 days, 10 days, 11 days, 12 days, 13 days, or 14 days. In an embodiment of the disclosure, baseline activity is monitored or measured for about 7 days. In an embodiment of the disclosure, baseline activity level is monitored or measured for a period of about 6 hours, 7 hours, 8 hours, 9 hours, 10 hours, 11 hours, 12 hours, 13 hours, 14 hours 15 hours, 16 hours 17 hours, 18 hours, 19 hours, 20 hours, 21 hours, 22 hours, 23 hours, or 24 hours per day. In an embodiment of the disclosure, baseline activity is monitored or measured while the subject is awake. In an embodiment of the disclosure, baseline activity is monitored or measured while the subject is asleep. In an embodiment of the disclosure, baseline activity is monitored or measured during hours in which the subject is awake and asleep.

In one embodiment, activity levels are improved as compared with a baseline activity level. In one embodiment, activity levels are improved by about 1% to about 50%. In another embodiment, activity levels are improved by about 1% to about 25% as compared with a baseline activity level. In another embodiment, activity levels are improved by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% as compared with a baseline activity level. In another embodiment, activity levels are improved by about 10%, 15%, 20%, 25%, 30%, 35%, 40%, 45%, or 50% as compared with a baseline activity level.

In another embodiment of the disclosure, activity levels are maintained as compared with a baseline activity level. In another embodiment, activity levels do not decrease as compared with a baseline activity level. In another embodiment, activity levels decline less over time in treated patients than untreated or placebo patients. In one embodiment, activity levels decline by about 5% in treated patients, while activity levels decline by about 20% or more for placebo or untreated patients.

In an embodiment of the disclosure, a subject wears an actigraphy monitor on the non-dominant arm. Wrist acceleration is continually measured by the monitor. The monitor records tri-axial acceleration at 30 Hz. An algorithm converts acceleration measurements into minute-by-minute activity counts. Each minute is classified at an activity level based on established and validated cutpoints. Algorithms can also determine wear time, calories, and other parameters. Daily activity data is converted into weekly activity levels to allow data comparison. Predetermined filters are utilized to ensure that only compliant data is analyzed. Such filters may include a minimum number of “wear awake” minutes (e.g., at least 600 minutes) to ensure compliance and having at least three compliant days for a complaint week. Filters may be based on industry standards used for actigraphy analysis.

Counts can be converted into activity levels in multiple ways. For example, the average of the counts provides a direct measure of physical activity. Each minute of the day can be converted into an activity intensity allowing the amount of time in sedentary, light, moderate, and vigorous activities to be determined as shown in Table A:

TABLE A
Physical Activity Parameter Examples

| Activity Intensity | Example Activities | Average Baseline Levels |
|---|---|---|
| Sedentary | Lying | 450 mins (7.5 hours) |
| (<100 counts) | Standing | |
| (<1.5 MET) | Computer Work | |
| Light | Washing Dishes | 491 mins (8.2 hours) |
| (100-1951 counts) | Washing Windows | |
| (1.6-3.0 MET) | Vacuuming | |
| Moderate | Walking | 63 mins (1.1 hours) |
| (1952-5724 counts) | Ascending/descending stairs | |
| (3.1-6.0 MET) | Lawn mowing | |
| Vigorous | Slow/fast running | 0 min |
| (>5724 counts) | Intense sports | |
| (>6.0 MET) | | |

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: A Phase-2 Trial of INOpulse in Patients with Sarcoidosis Associated Pulmonary Hypertension (SAPH) Requiring Supplemental Oxygen

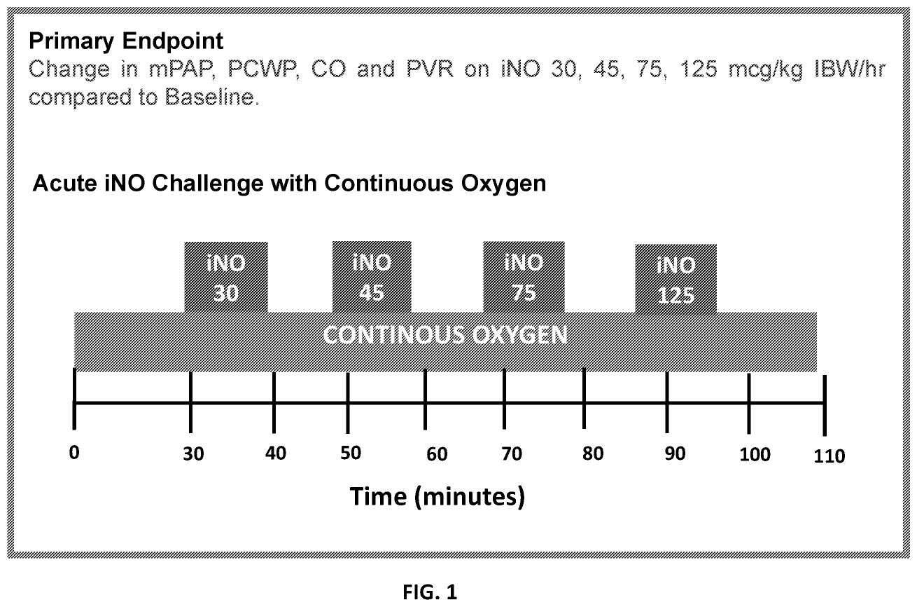

Patients with sarcoidosis may develop pulmonary hypertension (PH) which is associated with an impaired quality of life and limitations on activities of daily living. The Phase 2 trial, was designed to determine the safety and clinical efficacy of pulsed iNO, a potent and well-established vasodilator, on sarcoidosis-associated with PH patients, as well as to evaluate the safe and effective dose of iNO in subjects on long term oxygen therapy (LTOT) with possible or definite pulmonary hypertension associated with sarcoidosis. FIG. 1 shows an exemplary study design.

Sarcoidosis patients with PH on prior right heart catherization (RHC) or on echocardiography were enrolled in a two-part open label study. Patients underwent RHC, to establish baseline hemodynamics including mean pulmonary arterial pressure (mPAP), cardiac output (CO), pulmonary wedge pressure (PCWP) with derivation of pulmonary vascular resistance (PVR).

Patients were administered acute escalating doses of pulsatile iNO via an INOpulse device starting at 30 mcg/kg ideal body weight/hr (iNO30) and increasing up to 125 mcg/kg ideal body weight/hr (iNO125) to assess the impact of each dose level on hemodynamic parameters. Patients were treated with iNO for 10 minutes per dose with a 10-minute washout between doses.

Each dose level was followed by a washout period. All hemodynamic wave forms were reviewed by a blinded hemodynamic core lab for determination of end-exhalation PAP and PCWP. After the acute vasodilator study, subjects were eligible to participate in a long-term open label extension study at the dose of iNO found to be most beneficial by the RHC measurements, as determined by the investigator.

Tables 1A and 1B illustrate the subject demographics of the eight patients enrolled in the study.

TABLE 1A
Patient Baseline Demographics

| Patient | Age | | | | Weight | Height |
|---|---|---|---|---|---|---|
| Number | (years) | Sex | Race | Ethnicity | (kg) | (cm) |
| PF002-01- | 65 | Female | White | Not Hispanic | 99 | 156 |
| 002-001 | | | | or Latino | | |
| PF002-01- | 69 | Female | White | Not Hispanic | 62 | 157 |
| 002-002 | | | | or Latino | | |
| PF002-01- | 50 | Female | Black or African | Not Hispanic | 100 | 175 |
| 002-003 | | | American | or Latino | | |
| PF002-01- | 39 | Male | Other | Hispanic or | 8.5 | 158 |
| 005-004 | | | | Latino | | |
| PF002-01- | 59 | Male | Black or African | Not Reported | 102 | 174 |
| 005-005 | | | American | | | |
| PF002-01- | 71 | Female | Black or African | Not Hispanic | 68 | 157 |
| 007-001 | | | American | or Latino | | |
| PF002-01- | 71 | Male | White | Not Hispanic | 98 | 172 |
| 07-003 | | | | or Latino | | |
| PF002-01- | 6.5 | Female | Black or African | Not Hispanic | 102 | 160 |
| 007-005 | | | American | or Latino | | |
| MEDIAN | 65.0 | 37.5% | 50% White | 75% Not | 98.5 | 159.0 |
| | | Male | | Hispanic or | | |
| | | | | Latino | | |
| IQR | 12.8 | | | | 19.8 | 15.5 |

TABLE 1B
Patient Demographics

| | |
|---|---|
| Age (years) | 61.1 (11.37) |
| Male (%) | 37.5% |
| *FEV1 (% | 50% (11%) |
| Predicted) | |
| *FVC (% | 64% (18%) |
| Predicted) | |
| *DLCO (% | 35% (11%) |
| Predicted) | |
| *LTOT (L/min) | 3.0 (1.1%) |

Mean (Standard Deviation) reported based on available preliminary data. *7 of 8 data points available at the time of analysis.

Table 2 illustrates the baseline hemodynamic parameters.

TABLE 2
Baseline Hemodynamic Parameters

| | PVR | mPAP | CO | PCWP |
|---|---|---|---|---|
| Patient Number | (dynesec*cm-5) | (mmHg) | (L/min) | (mmHg) |
| PF002-01-002-001 | 284 | 38.3 | 6.6 | 15 |
| PF002-01-002-002 | 211 | 28.3 | 7.0 | 10 |
| PF002-01-002-003 | 133 | 37.3 | 6.2 | 27 |
| PF002-01-005-004 | 245 | 35.3 | 5.7 | 18 |
| PF002-01-005-005 | 648 | 46.7 | 4.0 | 14 |
| PF002-01-007-001 | 444 | 36.7 | 4.8 | 10 |
| PF002-01-007-003 | 541 | 37.0 | 3.6 | 13 |
| PF002-01-007-005 | 375 | 40.3 | 6.1 | 12 |
| MEDIAN | 329.4 | 37.2 | 5.9 | 13.5 |
| IQR | 231.7 | 2.5 | 1.7 | 4.3 |

Table 3 illustrates the change from baseline hemodynamic parameters.

| | Baseline | iNO30 | iNO45 | iNO75 | iNO125 |
|---|---|---|---|---|---|
| PVR | 329.4 | −19.2 (39.0) | −48.4 (84.6) | −68.0 (92.7) | −85.8 (67.1) |
| (dyne*sec*cm- | | −7.0% (10.6%) | −19.7% (18.3° A) | −15.3% (37.7%) | −28.7% (20.8%) |
| 5) | | | | | |
| mPAP | 37.2 | −2.7 (2.8) | −4.0 (5.2) | −2.3 (3.9) | −3.0 (3.5) |
| (mmHg) | | −7.1% (10.0%) | −10.6% (15.8%) | −6.3% (13.2%) | −6.4% (11.0%) |
| CO | 5.9 | −0.1 (0.6) | 0.0 (0.3) | 0.0 (0.4) | 0.2 (0.8) |
| (L/min) | | −2.5% (11.2) | 1.0% (6.5%) | 0.1% (6.1%) | 4.2% (14.6%) |
| PCWP | 13.5 | −1.0 (4.3) | 0.0 (2.5) | 1.5 (4.3) | 0.0 (4.0) |
| (mmHg) | | −4.1% (29.1%) | 0.0% (20.5%) | 10.6% (29.5%) | 0.0% (30.7%) |

During the Treatment Visit of Part 1 of the study, subjects were admitted to the cardiac catheterization suite/ICU/CCU and a Swan Ganz Catheter was inserted, and subjects were monitored during acute exposure to iNO at 30, 45, 75, 125 mcg/kg IBW/hr. Hemodynamic changes were monitored during this part of the study. The primary endpoint for Part 1 of the study was to assess the change in mean pulmonary artery pressure (mPAP), pulmonary capillary wedge pressure (PCWP), cardiac output (CO) and pulmonary vascular resistance (PVR) on iNO 30, 45, 75, 125 mcg/kg IBW/hr compared to baseline. The Safety Endpoint includes incidence and severity of treatment emergent adverse events (AEs), including those related to device deficiency, symptoms that may be due to rebound associated with acute withdrawal of iNO: systemic arterial oxygen desaturation, hypoxemia, bradycardia, tachycardia, systemic hypotension, shortness of breath, near-syncope, and syncope, and change in oxygen saturation and vital signs.

All 8 PH-Sarc patients were dose escalated to receive at least 75 mcg/kg and 7 were titrated to highest dose per protocol of 125 mcg/kg. As determined by the blinded core lab, all 8 patients demonstrated decreases in mPAP and PVR across the doses of iNO from iNO30 to iNO125. The baseline median mPAP for the group was 37.2 mmHg, which decreased by 6-10% across the doses of iNO30 to iNO125. The baseline median PVR for the group was 329 dyne*sec*cm-5, with the iNO45 dose demonstrating a median decrease of 20% (−54% to +22%). Increasing the dose to iNO125 dose resulted in median decrease of 29% (−43% to −5%; p=0.02 vs. baseline and prior dose). No adverse events or serious adverse events related to iNO were reported.

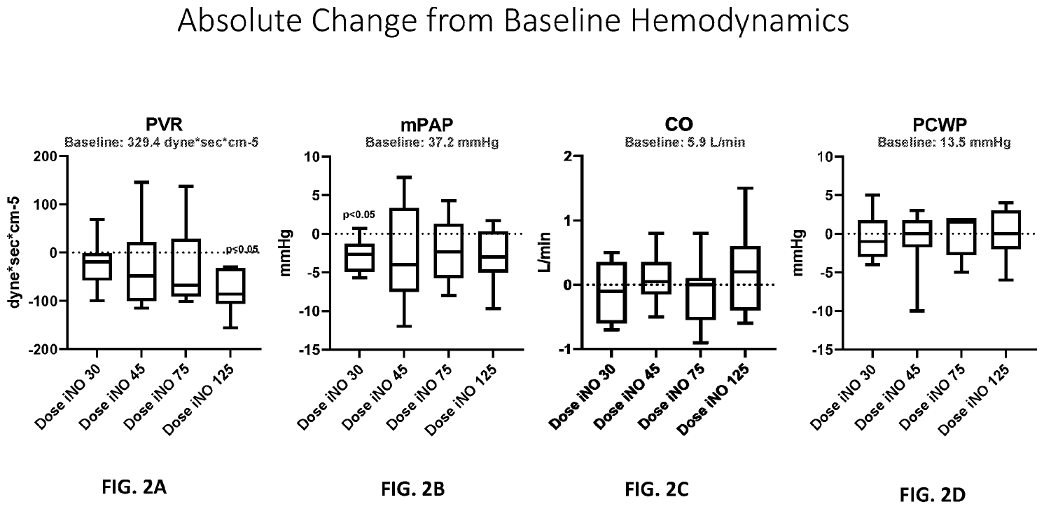

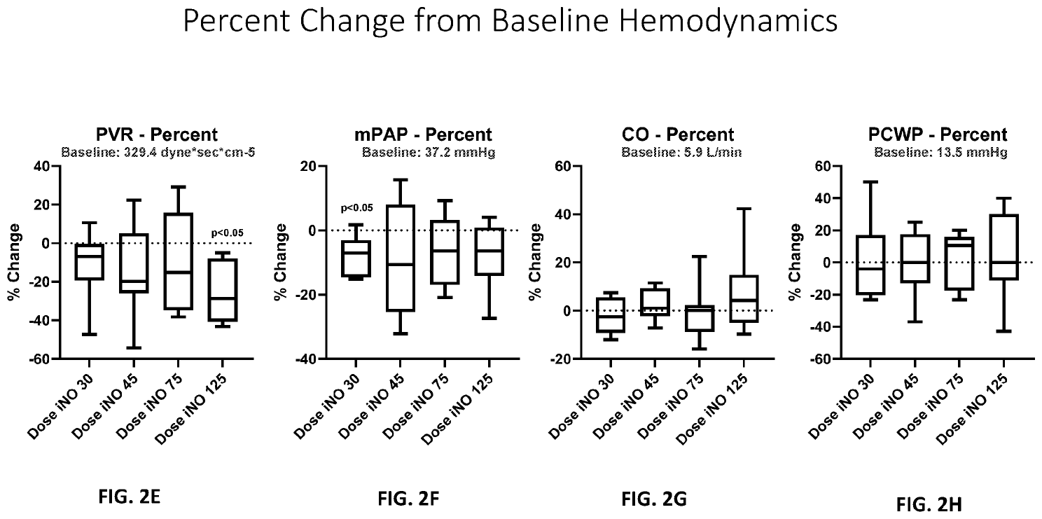

The changes in hemodynamics were determined for absolute change (as shown in FIGS. 2A-2D) and percent change (as shown in FIGS. 2E-2H) from baseline in PVR, mPAP, CO and PCWP. The analysis depicted illustrates the change from baseline (box & whisker plot provides median, IQR, and min/max). Statistical analysis based on Wilcoxon Log Rank Test was assessed between baseline and each dose as well as each dose and each prior dose and no correction was conducted for multiplicity. All 8 subjects demonstrated a decrease in mPAP and PVR across the dose ranges with mPAP reaching statistical significance for the iNO30 dose as compared to baseline and PVR reaching statistical significance at the iNO125 dose compared to both baseline as well as the prior dose of iNO75. CO and PCWP remained stable across all doses and iNO was well-tolerated across all the doses. Median mPAP reduction ranged from 6.3% to 10.6%, with the IQR ranging from 10% to 15.8%. Reduction in mPAP reached statistical significance for the iNO30 dose (p<0.05), however, due to the small data set, it did not reach the level of statistical significance for change from baseline or prior dose with any of the remaining doses. Cardiac output values remained stable across all doses, with no significant change noted at any dose of iNO.

Median reduction in PVR ranged from 7.0% to 28.7% with the IQR ranging from 10.6% to 37.7%. No dose was determined to be clearly superior given the overlap of the interquartile range amongst the doses and due to the small sample size, however statistically significant reduction in PVR was demonstrated with the iNO125 dose compared to baseline (p<0.05) as well as with the prior dose of iNO75.

While preferred embodiments of the disclosure are shown and described herein, such embodiments are provided by way of example only and are not intended to otherwise limit the scope of the disclosure. Various alternatives to the described embodiments of the disclosure may be employed in practicing the disclosure.

What is claimed is:

1. (canceled)

2. A method for treating pulmonary hypertension associated with sarcoidosis (PH-SARC) in a patient in need thereof, the method comprising administering inhaled nitric oxide (iNO) to the patient, wherein the iNO is delivered in a pulsatile manner by:
(a) detecting a breath pattern in the patient including a total inspiratory time of a single breath;
(b) correlating the breath pattern with an algorithm to calculate a timing of administration of a dose of nitric oxide; and
(c) delivering the nitric oxide to the patient in a pulsatile manner over a portion of the total inspiratory time.

3. The method of claim 2, wherein detecting a breath pattern includes a use of at least one trigger selected from a breath level trigger and a breath slope trigger.

4. The method of claim 2, wherein the algorithm uses one or both of a threshold sensitivity and a slope algorithm, wherein the slope algorithm detects a breath when a rate of pressure drop reaches a predetermined threshold.

5. The method of claim 2, wherein the treatment further includes reducing pulmonary vascular resistance (PVR) and/or reducing mean pulmonary artery pressure (mPAP) compared to baseline levels.

6. The method of claim 5, wherein pulmonary vascular resistance (PVR) is reduced by at least about 5%, about 10%, about 15%, about 20%, about 25%, about 30%, about 35%, about 40%, about 45%, or about 50% or more compared to baseline levels, optionally at least about 20%.

7. The method of claim 5, wherein mean pulmonary artery pressure (mPAP) is reduced by at least about 5%, about 10%, about 15%, about 20%, about 25%, about 30%, about 35%, about 40%, about 45%, or about 50% or more compared to baseline levels, optionally at least about 10%.

8. The method of claim 2, the method further comprising maintaining resting cardiac output (CO) and/or maintaining pulmonary capillary wedge pressure (PCWP) compared to baseline levels.

9. (canceled)

10. (canceled)

11. (canceled)

12. (canceled)

13. The method of claim 2, wherein the patient is undergoing long term oxygen therapy (LTOT).

14. The method claim 2, wherein the inhaled nitric oxide is administered at a dose in a range of from about 20 mcg/kg IBW/hr to about 150 mcg/kg IBW/hr.

15. (canceled)

16. (canceled)

17. (canceled)

18. A method for reducing pulmonary vascular resistance (PVR) or mean pulmonary arterial pressure (mPAP) in a patient having or at risk of having pulmonary hypertension associated with sarcoidosis (PH-SARC) in a patient in need thereof, the method comprising administering inhaled nitric oxide (iNO) to the patient, wherein the nitric oxide is delivered in a pulsatile manner by:
(a) detecting a breath pattern in the patient including a total inspiratory time of a single breath;
(b) correlating the breath pattern with an algorithm to calculate a timing of administration of a dose of nitric oxide; and
(c) delivering the nitric oxide to the patient in a pulsatile manner over a portion of the total inspiratory time.

19. (canceled)

20. The method of claim 18, wherein detecting a breath pattern includes a use of at least one trigger selected from a breath level trigger and a breath slope trigger.

21. The method of claim 18, wherein the algorithm uses one or both of a threshold sensitivity and a slope algorithm, wherein the slope algorithm detects a breath when a rate of pressure drop reaches a predetermined threshold.

22. The method of claim 18, wherein pulmonary vascular resistance (PVR) is reduced by at least about 5%, about 10%, about 15%, about 20%, about 25%, about 30%, about 35%, about 40%, about 45%, or about 50% or more compared to baseline levels, optionally at least about 20%.

23. The method of claim 18, wherein mean pulmonary artery pressure (mPAP) is reduced by at least about 5%, about 10%, about 15%, about 20%, about 25%, about 30%, about 35%, about 40%, about 45%, or about 50% or more compared to baseline levels, optionally at least about 10%.

24. The method of claim 18, the method further comprising maintaining resting cardiac output (CO) and/or maintaining pulmonary capillary wedge pressure (PCWP) compared to baseline levels.

25. (canceled)

26. (canceled)

27. (canceled)

28. (canceled)

29. The method of claim 18, wherein the patient is undergoing long term oxygen therapy (LTOT).

30. The method claim 18, wherein the inhaled nitric oxide is administered at a dose in a range of from about 20 mcg/kg IBW/hr to about 150 mcg/kg IBW/hr.

31. (canceled)

32. (canceled)

33. (canceled)

34. The method of claim 18, wherein the patient is at low risk, intermediate risk, or high risk of having and/or developing pulmonary hypertension (PH).

35. The method of claim 18, wherein the method further includes preventing a decline, maintaining, or improving one or more activity levels in the patient.

36. The method of claim 35, wherein the one or more activity levels are selected from overall activity, non-sedentary activity, moderate activity, moderate to vigorous physical activity (MVP A), steps, calories, metabolic equivalent units (MET), sleep, heart rate, oxygen saturation, calories burned, six-minute walk distance (6MWD) test, and other types of activity and/or daily activity parameters.

37.-55. (canceled)
